(19) United States
(12) Patent Application Publication
(10) Pub. No.: US 2024/0115643 A1
(43) Pub. Date: Apr. 11, 2024

(54) COMPOSITIONS AND METHODS FOR THE TREATMENT OF SKIN CONDITIONS

(71) Applicant: Nancy Franklin, Oakland, CA (US)

(72) Inventor: Nancy Franklin, Oakland, CA (US)

(21) Appl. No.: 18/484,441

(22) Filed: Oct. 10, 2023

Related U.S. Application Data
(60) Provisional application No. 63/378,903, filed on Oct. 10, 2022.
(60) Provisional application No. 63/387,300, filed on Dec. 14, 2022.
(60) Provisional application No. 63/584,158, filed on Sep. 20, 2023.

(51) Int. Cl. A61K 36/31; A61K 9/00; A61K 31/164; A61K 31/575; A61K 36/28

(52) U.S. Cl. CPC A61K 36/31; A61K 9/0014; A61K 31/164; A61K 31/575; A61K 36/28

(57) ABSTRACT

Topical compositions formulated for application to the skin, methods of preparing the topical compositions, and methods of administering the topical composition to a subject are described. The compositions can include one or more components selected from a Moringa oleifera extract, ceramide, free fatty acid, sterol, and silk. The method includes administering the topical composition to a subject to treat a skin disorder such as radiation dermatitis or erythema.

### CROSS-REFERENCE TO RELATED APPLICATIONS

This application claims the benefit of priority under 35 U.S.C. § 119(e) of U.S. Provisional Application Ser. No. 63/378,903 filed on Oct. 10, 2022; U.S. Provisional Application Ser. No. 63/387,300 filed on Dec. 14, 2022; U.S. Provisional Application Ser. No. 63/584,158 filed on Sep. 20, 2023, the contents of which are relied upon and incorporated herein by reference in their entireties.

### FIELD

The present disclosure generally relates to topical compositions, methods of preparing the topical compositions, and methods of treating a subject in need thereof with the topical compositions; and more particularly, to compositions formulated for topical administration, methods of preparing the compositions for topical administration, and treating a subject having a skin condition with the topical compositions.

### BACKGROUND

Dermatitis is inflammation of the skin. Dermatitis can vary in duration from short bouts to long-term cases and can involve a small area of skin or cover the entire body. There are several types of dermatitis. Atopic dermatitis, for example, is a long-term type of dermatitis that causes itchy, red, swollen, and cracked skin. The pathogenesis of atopic dermatitis is not fully understood but is believed to involve complex interactions of the subject's genes, immune system, and environment. Radiation dermatitis, as another example, is associated with prolonged exposure to ionizing radiation and occurs in patients receiving radiation therapy. The condition manifests itself in localized red patches of skin, or burns. There are no known cures for atopic or radiation dermatitis, and treatment goals usually focus on controlling and minimizing the symptoms. Additional therapeutic compositions for the treatment of dermatitis are needed.

### SUMMARY

In various embodiments, a composition is provided. In some embodiments, the composition formulated for topical application, the composition comprising: a Moringa oleifera extract in an amount ranging from 0.1-6.0%; a ceramide in an amount ranging from 0.1-1.0%; a free fatty acid in an amount ranging from 0.05-8%; a sterol in an amount ranging from 0.01-1.0%; and a silk in an amount ranging from 0.05-5.0%; wherein each respective amount is based on weight percent.

In some embodiments, the composition further comprises a calendula flower extract in an amount ranging from 0.05-3.0%.

In some embodiments, the ceramide is a Triticum vulgare (wheat) seed extract.

In some embodiments, the sterol is cholesterol or phytosterol.

In some embodiments, the free fatty acid is from an extract of sunflower oil or safflower oil. In some embodiments, the free fatty acid is linoleic acid, palmitic acid, stearic acid, or a combination thereof.

In some embodiments, the composition further comprises a mixture of Moringa oleifera, Eruca sativa, Brassica oleracea var. Sabellica, and Brassica oleracea var. Italica extract in an amount ranging from 1.0-10.0%, wherein the mixture is a distinct component with respect to the Moringa oleifera extract.

In some embodiments, the composition further comprises green tea extract in an amount ranging from 0.01-2.0%.

In some embodiments, the composition further comprises Triticum vulgare (wheat) seed extract in an amount ranging from 0.1-0.7%.

In some embodiments, the composition further comprises glycyrrhizinic acid in an amount ranging from 0.2-3.0%.

In some embodiments, the composition further comprises Avena sativa (oat) kernel extract in an amount ranging from 0.2-3.0%.

In some embodiments, the composition further comprises water in an amount ranging from 25-50%; an aloe vera in an amount ranging from 5-20%; a jojoba oil in an amount ranging from 5-18%; a sodium cocycl isethionate in an amount ranging from 2-11%; a glycerin in an amount ranging from 1-10%; a gum in an amount ranging from 0.2-3%; Avena sativa (oat) kernel extract in an amount ranging from 0.2-3.0%; a citric acid in an amount ranging from 0.09-2%; a coconut oil in an amount ranging from 0.9-6%; a mixture of benzyl alcohol and dehydroacetic acid in a combined amount ranging from 0.5-3.0%; a squalane in an amount ranging from 0.2-3%; tocopheryl acetate in an amount ranging from 0.2-3.0%; and a mixture of cetearyl alcohol and sorbitan olivate in a combined amount ranging from 0.2-6.0%.

In some embodiments, the silk is vegan silk polypeptide.

In some embodiments, the composition increases Nuclear factor erythroid-2-related factor 2 (Nrf2) activation in human keratinocytes when applied topically to a human subject.

In some embodiments, a topical composition formulated for application to human skin is provided, the topical composition comprising: Moringa oleifera extract in an amount ranging from 1.0-10.0%; calendula flower extract in an amount ranging from 0.05-1.0%; silk in an amount ranging from 0.09-3.0%; and phytosterol in an amount ranging from 0.05-1.0%; a mixture of Moringa oleifera, Eruca sativa, Brassica oleracea var. Sabellica, and Brassica oleracea var. Italica extract in an amount ranging from 1.0-10.0%; green tea extract in an amount ranging from 0.01-2%; wherein the amounts are based on weight percent.

In some embodiments, the composition further comprises Triticum vulgare (wheat) seed extract in an amount ranging from 0.1-0.7%; glycyrrhetic acid in an amount ranging from 0.2-3%; Avena sativa (oat) kernel extract in an amount ranging from 0.2-3%; water in an amount ranging from 25-50%; aloe vera in an amount ranging from 5-20%; jojoba oil in an amount ranging from 5-18%; sodium cocycl isethionate in an amount ranging from 2-11%; glycerin in an amount ranging from 1-10%; xanthan gum in an amount ranging from 0.2-3%; sunflower oil in an amount ranging from 0.5-5%; citric acid in an amount ranging from 0.09-2%; coconut oil in an amount ranging from 0.9-6%; benzyl alcohol and dehydroacetic acid in a combined amount ranging from 0.5-3%; squalane in an amount ranging from 0.2-3%; tocopheryl acetate in an amount ranging from 0.2-3%; and cetearyl alcohol and sorbitan olivate in a combined amount ranging from 0.2-6%.

In various embodiments, a topical composition formulated for application to human skin is provided, the topical composition comprising: a Moringa oleifera extract in an amount ranging from 1.0-10.0%; a silk in an amount ranging from 0.09-3.0%; a sterol selected from the group of cholesterol and phytosterol in an amount ranging from 0.05-1.0%; a mixture of Moringa oleifera, Eruca sativa, Brassica oleracea var. Sabellica, and Brassica oleracea var. Italica extract in an amount ranging from 1.0-10.0%, wherein the mixture is a distinct component with respect to the Moringa oleifera extract; a Triticum vulgare (wheat) seed extract in an amount ranging from 0.1-0.7%; a sunflower oil in an amount ranging from 0.5-5%; a green tea extract in an amount ranging from 0.01-2%; a glycyrrhizinic acid in an amount ranging from 0.2-3%; an Avena sativa (oat) kernel extract in an amount ranging from 0.2-3%; water in an amount ranging from 25-50%; an aloe vera in an amount ranging from 5-20%; a jojoba oil in an amount ranging from 5-18%; a sodium cocycl isethionate in an amount ranging from 2-11%; a glycerin in an amount ranging from 1-10%; a xanthan gum in an amount ranging from 0.2-3%; a citric acid in an amount ranging from 0.09-2%; a coconut oil in an amount ranging from 0.9-6%; a benzyl alcohol and dehydroacetic acid in a combined amount ranging from 0.5-3%; a squalane in an amount ranging from 0.2-3%; a tocopheryl acetate in an amount ranging from 0.2-3%; and a mixture of cetearyl alcohol and sorbitan olivate in a combined amount ranging from 0.2-6%; wherein each respective amount is based on weight percent.

In some embodiments, the Moringa oleifera extract is in an amount ranging from 2.0-7.0%; the silk is in an amount ranging from 0.7-1.0%; the sterol is in an amount ranging from 0.09-1.0%; the mixture of Moringa oleifera, Eruca sativa, Brassica oleracea var. Sabellica, and Brassica oleracea var. Italica extract is in an amount ranging from 1.5-6.0%; the green tea extract is in an amount ranging from 0.01-1.0%; the Triticum vulgare (wheat) seed extract is in an amount ranging from 0.1-0.7%; the glycyrrhizinic acid is in an amount ranging from 0.5-2.5%; the Avena sativa (oat) kernel extract is in an amount ranging from 0.5-2.5%; the water is in an amount ranging from 30-45%; the aloe vera is in an amount ranging from 8-18%; the jojoba oil is in an amount ranging from 8-15%; the sodium cocycl isethionate is in an amount ranging from 4-10%; the glycerin is in an amount ranging from 3-8%; the xanthan gum is in an amount ranging from 0.5-2.5%; the sunflower oil is in an amount ranging from 0.5-4%; the citric acid is in an amount ranging from 0.09-2%; the coconut oil is in an amount ranging from 1-7%; the mixture of benzyl alcohol and dehydroacetic acid is in a combined amount ranging from 0.5-2.5%; the squalane in an amount ranging from 0.5-2.5%; the tocopheryl acetate is in an amount ranging from 0.5-2.5%; and the mixture of cetearyl alcohol and sorbitan olivate is in a combined amount ranging from 1-5.5%; wherein each respective amount is based on weight percent.

In some embodiments, the Moringa oleifera extract is in an amount ranging from 3.0-6.0%; the silk is in an amount ranging from 0.8-1.5%; the sterol is in an amount ranging from 0.15-0.7%; the mixture of Moringa oleifera, Eruca sativa, Brassica oleracea var. Sabellica, and Brassica oleracea var. Italica extract is in an amount ranging from 2.0-5.0%; the green tea extract is in an amount ranging from 0.02-0.2%; the Triticum vulgare (wheat) seed extract is in an amount ranging from 0.2-0.5%; the glycyrrhizinic acid is in an amount ranging from 0.5-1.5%; the Avena sativa (oat) kernel extract is in an amount ranging from 0.5-1.5%; the water is in an amount ranging from 35-40%; the aloe vera is in an amount ranging from 11-17%; the jojoba oil is in an amount ranging from 10-14%; the sodium cocycl isethionate is in an amount ranging from 5-9%; the glycerin is in an amount ranging from 3-7%; the xanthan gum is in an amount ranging from 0.5-1.5%; the sunflower oil is in an amount ranging from 1-3%; the citric acid is in an amount ranging from 0.1-0.8%; the coconut oil is in an amount ranging from 2-5%; the mixture of benzyl alcohol and dehydroacetic acid is in a combined amount ranging from 0.5-1.5%; the squalane is in an amount ranging from 0.5-1.5%; the tocopheryl acetate is in an amount ranging from 0.5-1.5%; and the mixture of cetearyl alcohol and sorbitan olivate is in a combined amount ranging from 2-5%.

In some embodiments, the Moringa oleifera extract in an amount ranging from 3.0-6.0%; calendula extract in an amount ranging from 0.2-0.5%; silk in an amount ranging from 0.8-1.5%; and phytosterol in an amount ranging from 0.15-0.7%; a mixture of Moringa oleifera, Eruca sativa, Brassica oleracea var. Sabellica, and Brassica oleracea var. Italica extract in an amount ranging from 2.0-5.0%; green tea extract in an amount ranging from 0.02-0.2%; Triticum vulgare (wheat) seed extract in an amount ranging from 0.2-0.5%; glycyrrhetic acid in an amount ranging from 0.5-1.5%; Avena sativa (oat) kernel extract in an amount ranging from 0.5-1.5%; water in an amount ranging from 35-40%; aloe vera in an amount ranging from 11-17%; jojoba oil in an amount ranging from 10-14%; sodium cocycl isethionate in an amount ranging from 5-9%; glycerin in an amount ranging from 3-7%; xanthan gum in an amount ranging from 0.5-1.5%; sunflower oil in an amount ranging from 1-3%; citric acid in an amount ranging from 0.1-0.8%; coconut oil in an amount ranging from 2-5%; benzyl alcohol and dehydroacetic acid in a combined amount ranging from 0.5-1.5%; squalane in an amount ranging from 0.5-1.5%; tocopheryl acetate in an amount ranging from 0.5-1.5%; and cetearyl alcohol and sorbitan olivate in a combined amount ranging from 2-5%; wherein the amounts are based on weight percent.

In some embodiments, the composition further comprises a calendula flower extract in an amount ranging from 0.05-1.0%.

In various embodiments, a method of treating a skin disorder in a subject in need thereof is provided. In such embodiments, the method comprises topically administering to the subject a composition comprising: a Moringa oleifera extract in an amount ranging from 0.1-6.0%; a ceramide in an amount ranging from 0.1-1.0%; a free fatty acid in an amount ranging from 0.05-8%; a sterol in an amount ranging from 0.01-1.0%; and a silk in an amount ranging from 0.05-5.0%; wherein each respective amount is based on weight percent.

In some embodiments, the composition in the method further comprises a mixture of Moringa oleifera, Eruca sativa, Brassica oleracea var. Sabellica, and Brassica oleracea var. Italica extract in an amount ranging from 1.0-10.0%, wherein the mixture is a distinct component with respect to the Moringa oleifera extract; a green tea extract in an amount ranging from 0.01-2.0%; a glycyrrhizinic acid in an amount ranging from 0.2-3%; an Avena sativa (oat) kernel extract in an amount ranging from 0.2-3%; a water in an amount ranging from 25-50%; an aloe vera in an amount ranging from 5-20%; a jojoba oil in an amount ranging from 5-18%; a sodium cocycl isethionate in an amount ranging from 2-11%; a glycerin in an amount ranging from 1-10%; a xanthan gum in an amount ranging from 0.2-3%; a citric acid in an amount ranging from 0.09-2%; a coconut oil in an amount ranging from 0.9-6%; a mixture of benzyl alcohol and dehydroacetic acid in a combined amount ranging from 0.5-3%; a squalane in an amount ranging from 0.2-3%; an atocopheryl acetate in an amount ranging from 0.2-3%; and a mixture of cetearyl alcohol and sorbitan olivate in a combined amount ranging from 0.2-4.5%.

In some embodiments, the skin disorder in the method is dermatitis or erythema. In some embodiments, the skin disorder is radiation dermatitis. In some embodiments, the composition increases Nuclear factor erythroid-2-related factor 2 (Nrf2) activation in human keratinocytes when applied topically to a human subject.

In some embodiments, the administering step of the method is completed once per day, twice per day, or three times per day.

In some embodiments, the composition in the method is in the form of a cream or lotion.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings illustrate the various embodiments described herein and, together with the description, explain the principles and operations of the claimed subject matter.

### DETAILED DESCRIPTION

Reference will now be made in detail to the exemplary embodiment(s), examples of which is/are illustrated in the examples. Before describing the exemplary embodiments, it is noted the embodiments reside primarily in combinations of components and procedures related to the topical compositions and methods of using the topical compositions. Accordingly, the composition and method components have been represented where appropriate, showing only those specific details that are pertinent to understanding the embodiments of the present disclosure so as not to obscure the disclosure with details that will be readily apparent to those of ordinary skill in the art having the benefit of the description herein. The specific details of the various embodiments described herein are used for demonstration purposes only, and no unnecessary limitation or inferences are to be understood therefrom.

In various embodiments, a composition for topical administration, or topical composition, is provided. In some embodiments, the topical composition may be used to treat one or more skin disorders, including, for example, dermatitis. In some embodiments, the topical composition may be used to treat acute or chronic dermatitis. In some embodiments, the topical composition may be used to treat radiation dermatitis. In various embodiments, a method of administering the topical composition to a subject in need thereof is provided. In some embodiments, a method of treating acute or chronic dermatitis comprising the administration of the topical composition is provided. In some embodiments, a method of treating radiation dermatitis comprising the administration of the topical composition is provided.

As used herein, the term “topical application” refers to the application or spread of a composition onto the surface of keratinous tissue, including, for example, human skin. As used herein, the terms “topical composition,” “topical skin care composition,” and “topical skin composition” are used interchangeably and include compositions suitable for topical application on keratinous tissue. In some embodiments, the topical compositions are dermatologically acceptable in that they do not have undue toxicity, incompatibility, instability, allergic response, and the like, when applied to the skin. In some embodiments, the topical compositions are dermatologically acceptable in that they are sterile and therefore free of any microorganisms. In some embodiments, the topical compositions have a predetermined viscosity to avoid significant dripping or pooling after application to the skin.

As used herein, the term “skin” refers to the skin of all or one or more parts of the body, particularly human body, including the face, scalp, neck, chest, back, stomach, torso, legs, feet, underarms, hands, thighs, shins, arms, and labial mucosa.

As used herein, the term “comprising” is synonymous with “including” and “containing,” and intended to be inclusive or open-ended so as to not exclude additional, unrecited elements or steps. As used herein, the term “consisting of” is intended to be exclusive of any component or step not specified. As used herein, the term “consists essentially of” is intended to limit the scope to the specified components and steps and other components or steps that do not materially affect the compositions or methods. As used herein, the recitation of “comprising” in one embodiment should not be read to exclude an alternative embodiment having “consisting of” or “consisting essentially of” language. Unless explicitly stated, the following embodiments reciting “comprising” shall be read to provide support for alternative embodiments based on the terms “consists of” and “consisting essentially of.”

In various embodiments, the topical composition comprises one or more plant-derived components. In some embodiments, the topical composition comprises the parts and/or extracts from one or more plants. In various embodiments, the topical composition comprises one or more isolated and/or purified plant-derived components.

In some embodiments, the topical composition comprises the parts and/or extracts or isolated compounds from one or more species of the family Moringaceae, including the genus Moringa and the species thereof, namely, M. oleifera, M. arborea, M. borziana, M. concanensis, M. drouhardii, M. hildebrandtii, M. longituba, M. ovalifolia, M. peregrina, M. pygmaea, M. rivae, M. ruspoliana, and M. stenopetala. As used herein, and unless further specified, the term “moringa” includes all species of the genus Moringa; all varieties, cultivars, ecotypes, landraces, pure line varieties and pure line selections, indeterminate or standard forms and dwarf forms of Moringa; and all extracts, oils, and any whole or partial plant parts, including leaves, seed, roots, stems fruit, and flowers of Moringa.

Extracts from one or more species of the family Moringaceae can be obtained by extraction methods known to those of ordinary skill in the art. In some embodiments, the extract can be obtained from one or more portions of the plant, including, for example, the whole fruit, whole vegetable, whole plant, whole tree, whole bush, seed, peel, fruit, stem, bark, leaf, root, flower, petal, bulb, etc.

In various embodiments, the topical composition comprises one or more moringa components. In various embodiments, the topical composition comprises one or more isolated and/or purified moringa components. In some embodiments, for example, the topical composition comprises from about 0.001% to about 50%, from about 0.01% to about 40%, from about 0.1% to about 30%, from about 1% to about 20%, from about 2% to about 10%, or from about 3% to about 5%, by weight, respectively, of one or more of moringa components including M. oleifera leaf extract, M. oleifera seed extract, M. oleifera root extract, M. oleifera stem extract, M. oleifera oil, M. oleifera leaf powder. In some embodiments, the total amount for each respective component of the one or more moringa components in the topical compositions is about 1.0 wt. %, 1.5 wt. %, 2.0 wt. %, 2.5 wt. %, 3.0 wt. %, 3.5 wt. %, 4.0 wt. %, 4.5 wt. %, 5 wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, or 10 wt. %.

Extracts of the one or more moringa components may be combined in any suitable concentration ratio when forming the topical composition. In some embodiments, for example, the topical composition comprises two moringa components. In such embodiments, each of the respective moringa components can be combined in any suitable ratio. For example, the two moringa components can be combined to form a composition at a ratio ranging from about 0.1:500, 1:400, 1:100, 1:50, 1:10, 1:5, 1:2, 1:1, etc. (first component:second component).

In various embodiments, the topical composition comprises calendula. In various embodiments, the topical composition comprises isolated and/or purified calendula. In some embodiments, the topical composition comprises moringa and calendula. In some embodiments, the topical compositions comprises from about 0.001% to about 50%, from about 0.01% to about 40%, from about 0.1% to about 30%, from about 0.2% to about 2.0%, or from about 0.3% to about 0.8%, by weight, of calendula flower extract, calendula seed oil, calendula seed extract, calendula flower oil, calendula root extract. In some embodiments, the total amount of the calendula in the topical compositions is about 0.1 wt. %, 0.2 wt. %, 0.3 wt. %, 0.4 wt. %, 0.5, wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.5 wt. %, 3.0 wt. %, 3.5 wt. %, 4.0 wt. %, or 5 wt. %.

In some embodiments, the topical composition comprises silk. In this context, the term “silk” includes silk and/or a subcomponent of silk, either of which being derived from an organism or synthetically prepared. In some embodiments, the silk may be derived from one or more sources, including, for example, silkworm silk, hydrolyzed silk, silk fibroin, regenerated silk fibroin (e.g., liquid silk that can be used to generate fibers, films, hydrogels, etc.), silk-like proteins, silkelastin hybrid peptides, spider silk derived proteins, vegan silk polypeptides, and marine animal derived silk (e.g., squid, mussels, barnacles, oysters, sea anemones, bivalve mollusks), including byssus and byssal threads, hymenopteran silk (from, e.g., bees, ants, hornets), green lacewing silk (Mallada signata), honeybee silk, raspy cricket silk (Hyalogryllacris species 9, Apotrecheus illawarra), webspinner silk (Aposthonia gurneyi), caddisfly silk, and weaver ant silk. In some embodiments, the silk includes fibroin and/or sericin. In some embodiments, the silk is a protein polymer (e.g., a recombinant protein polymer derived from spiker silk), including, for example, the B-SILK protein provided by Bolt Threads, Inc. (CA). See, e.g., U.S. Pat. Nos. 9,9963,554; 10,988,515; 10,906,947; 10,647,975; 10,435,516; 10,035,886; 11,725,030; 11,634,729; 11,192,982; 11,208,736; 11,214,785; 11,306,127; 11,370,815; 11,447,532; the contents of which are incorporated herein by reference in their entireties without limitation. In some embodiments, the topical composition comprises moringa and silk. In some embodiments, the composition comprises moringa, calendula, and silk. In some embodiments, the topical compositions comprises from about 0.001% to about 50%, from about 0.01% to about 40%, from about 0.03% to about 15%, from about 0.05% to about 10%, from about 0.2% to about 5%, or from about 0.3% to about 1%, by weight, of silk. In some embodiments, the total amount of the silk component in the topical compositions is about 0.03 wt. %, 0.05 wt. %, 0.7 wt. %, 0.1 wt. %, 0.2 wt. %, 0.3 wt. %, 0.4 wt. %, 0.5, wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.5 wt. %, 3.0 wt. %, 3.5 wt. %, 4.0 wt. %, 5 wt. %, 6 wt. %, 7 wt. %, 8 wt. %, 9 wt. %, or 10 wt. %.

In some embodiments, the silk component provides a safe, biodegradable substitute for existing silicone elastomers, such as dimethicone, and petroleum-derived carbomers (common components in existing formulations). In some embodiments, the compositions are free of petrochemicals and siloxanes/silicones. In some embodiments, the silk can function to form a film on the skin of the subject, which creates a semipermeable breathable film barrier. In such embodiments, the silk molecules are large enough (70,000 Daltons) to be not absorbed through the skin, and therefore remain on the surface of the skin where their fibers join to create the film. In some embodiments, the film is capable of blocking pollution that delivers free radicals known to damage the skin barrier and therefore contribute to dry skin, aging, or atopic dermatitis flares. In some embodiments, the film reduces or prevents damage from UVA, UVB, and blue light. In some embodiments, the film functions to lock in the other components in the topical composition and to allow those components to remain in contact with the skin for longer periods. In such embodiments, the silk facilitates wound healing.

In various embodiments, the topical composition comprises green tea extract. In various embodiments, the topical composition comprises isolated and/or purified green tea extract. In some embodiments, the topical composition comprises moringa and green tea extract. In some embodiments, the topical composition comprises moringa, calendula, and green tea extract. In some embodiments, the topical composition comprises moringa, calendula, silk, and green tea extract. In some embodiments, the topical composition comprises from about 0.001% to about 50%, from about 0.01% to about 40%, from about 0.02% to about 2%, from about 0.01% to about 0.1%, or from about 0.02% to about 0.05%, respectively, by weight, of green tea extract. In some embodiments, the total amount of the green tea extract in the topical compositions is about 0.01 wt. %, 0.015 wt. %, 0.02 wt. %, 0.025 wt. %, 0.03 wt. %, 0.035 wt. %, 0.04 wt. %, 0.045 wt. %, 0.05, wt. %, 0.055 wt. %, 0.06 wt. %, 0.065 wt. %, 0.07 wt. %, 0.075 wt. %, 0.08 wt. %, 0.085 wt. %, 0.09 wt. %, 0.095 wt. %, or 1.0 wt. %.

In some embodiments, the green tea extract comprises a plurality of compounds, including polyphenols such as, e.g., epigallocatechin gallate (ECGC). The amount of green tea extract, or one or more specific compounds isolated and/or purified therefrom, can be modified based on the desired results.

In various embodiments, the topical composition comprises one or more ceramides. In some embodiments, the topical composition comprises moringa and one or more ceramides. In some embodiments, the topical composition comprises moringa, calendula, and one or more ceramides. In some embodiments, the topical composition comprises moringa, calendula, green tea extract, and one or more ceramides. In some embodiments, the topical composition comprises moringa, calendula, silk, green tea extract, and one or more ceramides.

The ceramides can be animal derived (e.g., bovine spinal cord), synthetic, or plant derived (“phytoceramides”). Unless further specified, the term “ceramides” includes all types of ceramides, that is, regardless of their origin.

In some embodiments, the topical composition comprises one or more animal derived epidermal ceramides, including ceramides found in human skin, such as: ceramide 1, ceramide 2, ceramide 3, ceramide 4, ceramide 5, ceramide 1A, ceramide 6 II, ceramide AP, ceramide EOP, ceramide EOS, ceramide NP, ceramide NG, ceramide NS, ceramide AS, and ceramide NS dilaurate. In some embodiments, the topical composition comprises one or more isolated and/or purified epidermal ceramides. In some embodiments, topical compositions comprises a plurality of epidermal ceramide compounds, including two, three, or more ceramides. In some embodiments, the respective ceramide compounds are isolated and/or purified. In some embodiments, for example, the topical composition includes ceramide 3, either alone or in combination with one or more other ceramides. In some embodiments, for example, the topical composition includes ceramide 3 and ceramide EOP, and optionally one or more additional isolated and/or purified ceramides.

In some embodiments, the topical composition comprises one or more plant derived phytoceramides. In some embodiments, topical compositions comprises a plurality of phytoceramides, including two, three, four, or more phytoceramides. In some embodiments, the topical composition comprises one or more isolated and/or purified phytoceramides. In some embodiments, the one or more phytoceramides comprise glucosylceramides (GlcCERs). In some embodiments, the phytoceramides are derived from, for example, rice, wheat, konjac, oats, soybeans, potatoes, sweet potatoes, maize, beets, or kidney beans.

In some embodiments, the topical composition comprises a 100% wheat-derived phytoceramide (e.g., Triticum vulgare (wheat) seed extract) (e.g., C+eramide), which is a combination of glycosylceramides (>50%) and DGDG (digalactosyl diglyceride) (>40%). In some embodiments, the Triticum vulgare (wheat) seed extract is gluten free. In some embodiments, the wheat-derived phytoceramide is isolated and/or purified.

In some embodiments, the total amount of the one or more ceramides in the topical composition is in the range of from about 0.1 wt. % to about 10 wt. %, from about 0.2 wt. % to about 5 wt. %, from about 0.1 wt. % to about 0.5 wt. %, or from about 0.2 wt. % to about 0.4 wt. %. In some embodiments, the total amount of the one or more ceramides in the topical compositions is about 0.1 wt. %, 0.2 wt. %, 0.3 wt. %, 0.4 wt. %, 0.5, wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, or 2.0 wt. % to 2.5 wt. %, 3.0 wt. %, 3.5 wt. %, 4.0 wt. %, or 5 wt. %.

In various embodiments, the topical composition comprises a sterol. In some embodiments, the sterol is cholesterol. In such embodiments, the cholesterol can be animal derived, synthetic, or derived from plants. In some embodiments, the topical composition comprises phytosterol. Like cholesterol, phytosterol can be incorporated into the stratum corneum lipid matrix and help repair a damaged epidermal barrier. In some embodiments, the cholesterol or phytosterol is isolated and/or purified.

In some embodiments, the topical composition comprises moringa and a cholesterol or phytosterol. In some embodiments, the topical composition comprises moringa, calendula, and cholesterol or phytosterol. In some embodiments, the topical composition comprises moringa, calendula, green tea extract, and cholesterol or phytosterol. In some embodiments, the topical composition comprises moringa, calendula, green tea extract, one or more ceramides, and cholesterol or phytosterol. In some embodiments, the topical composition comprises moringa, calendula, silk, green tea extract, one or more ceramides, and cholesterol or phytosterol.

In various embodiments, the topical composition comprises niacinamide (or nicotinamide). The niacinamide can be synthetic or derived from food. In some embodiments, the niacinamide is isolated and/or purified. In some embodiments, the total amount of niacinamide in the topical compositions is about 1.0 wt. %, 1.5 wt. %, 2.0 wt. %, 2.5 wt. %, 3.0 wt. %, 3.5 wt. %, 4.0 wt. %, 4.5 wt. %, 5 wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, or 10 wt. %.

In various embodiments, the topical composition comprises one or more free fatty acids. Each respective free fatty acid can be synthetic or derived from a natural product. In some embodiments, the free fatty acid is isolated and/or purified. Safflower and sunflower, for example, are sources of the essential fatty acid linoleic acid, the primary fatty acid in the lipid matrix of the stratum corneum. Other sources of linoleic acid, palmitic acid, or stearic acid, or a combination thereof may be sourced from one or more of grape seed oil, hemp oil, coconut oil, olive oil, cocoa butter, palm oil, avocado oil, rose hip oil, shea butter, sea buckthorn oil, sweet almond oil, borage oil, hempseed oil, and neem oil. In some embodiments, the total amount of free fatty acid in the topical compositions is about 1.0 wt. %, 1.5 wt. %, 2.0 wt. %, 2.5 wt. %, 3.0 wt. %, 3.5 wt. %, 4.0 wt. %, 4.5 wt. %, 5 wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 35 wt. %, 40 wt. %, 45 wt. %, 50 wt. %.

In various embodiments, the topical composition comprises phytosphingosine. The phytosphingosine can be synthetic, derived from an animal, or derived from a natural product (e.g., plant, fungi). In some embodiments, the phytosphingosine is isolated and/or purified.

In various embodiments, the topical composition comprises an aloe vera component, including aloe vera in the form of juice, gel, powder, oil, and/or extract. In some embodiments, the aloe vera component is isolated and/or purified.

In various embodiments, the topical composition comprises, consists of, or consists essentially of any number of combinations of the components described throughout this disclosure. The concentrations of any components within the compositions can vary. Notwithstanding the concentrations recited in the paragraphs above, in some embodiments the compositions may comprise, consist of, or consist essentially of, in final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99%, or any range derivable therein, of at least one of the components mentioned throughout this disclosure. The percentage can be calculated by weight or volume of the total composition. In some embodiments, the concentrations vary depending on the addition, substitution, and/or subtraction of one or more components in a respective composition.

In some embodiments, the topical composition is formulated for topical skin application one or more times per day. For example, in some embodiments, the topical composition is administered twice per day, three times per day, four times a day, etc. In some embodiments, the topical composition is storage stable and formulated for long-term storage (e.g., 3 or more years). In some embodiments, the topical composition is color stable.

In some embodiments, the viscosity of the topical composition is selected to achieve a desired result (e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000 cps, etc., as measured on a Brookfield Viscometer using a TC spindle at 2.5 rpm at 25° C.). In some embodiments, the topical composition has a predetermined viscosity to avoid significant dripping or pooling after application to the skin.

In some embodiments, the topical composition has a pH of 1, 2, 3, 4, 5, 6, 7, 8, 9, 10, 11, 12, 13, or 14. In some embodiments, the topical composition has a pH in the range of about 4 to about 8, or in the range of about 5 to about 7. In some embodiments, the topical composition has a pH in the range of about 5.5 to about 6.

In some embodiments, the topical composition comprises one or more carriers, excipients, diluents, stabilizers, lubricants, moisturizers, and/or solubilizers, such as those used for various routes of topical administration. In such embodiments, diluents and excipients such as fillers, extenders, binders, wetting agents, disintegrants, or surfactants, can be used.

Carriers and Formulations

In some embodiments, the topical composition comprises a dermatologically acceptable carrier (also referred to as a vehicle). Example carriers include water, glycerin, chlorphenesin, sodium metabisulfite, or a combination thereof.

In some embodiments, the topical composition can be incorporated into one or more types of carriers. Examples of suitable vehicles include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, and ointments or by other method or any combination of the forgoing as would be known to one of ordinary skill in the art. Variations and other appropriate vehicles would be apparent to one of ordinary skill in the art. In some embodiments, the concentrations and combinations of the compounds, components, and agents are selected such that the combinations are chemically compatible and do not form complexes that precipitate from the finished product.

In some embodiments, the topical composition can be used in any moisturizing creams, skin benefit creams and lotions, day lotions, gels, ointments, night creams. In some embodiments, the topical composition can be formulated as a leave-on or rinse-off product.

In some embodiments, the topical composition is formulated as a sprayable composition. In this context, the term sprayable means the composition is dispensed through a spray dispenser (e.g., hand-held spray dispenser). In some embodiments, the sprayable composition is useful for treating fragile and/or sensitive skin. In some embodiments, the topical composition can be continuously sprayed onto an area of the subject's skin. In this context, the term continuously sprayed refers to a formulation that provides any-angle spraying and uniform coverage. In some embodiments, the sprayable composition allows droplets of the topical composition to be dispersed to the skin without the need for excessive rubbing. In some embodiments, the topical composition is formulated for administration as a spray, aerosol, nebulizer solution, or the like, that can be applied to the skin of the subject. One of skill in the art would appreciate that containers and canisters having pumps or propellant (e.g., compressed air) can be used to dispense the sprayable topical composition.

In some embodiments, the topical composition can be encapsulated for delivery to a target area such as skin. Examples of encapsulation techniques include the use of liposomes, vesicles, and/or nanoparticles (e.g., biodegradable and non-biodegradable colloidal particles comprising polymeric materials in which the component is trapped, encapsulated, and/or absorbed; including, for example nanospheres and nanocapsules) that can be used as delivery vehicles to deliver the topical composition to skin.

Moisturizing Agents

In some embodiments, the topical composition comprises one or more moisturizing agents. Examples of moisturizing agents include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol. Further examples include acetylated lanolin, acetylated lanolin alcohol, alanine, algae extract, aloe barbadensis, aloe-barbadensis extract, aloe barbadensis gel, allantoin, althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, Calophyllum inophyllum (foraha) nut oil, candelilla (Euphorbia cerifera) wax, canola oil, caprylic/capric triglyceride, Cardamon (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, clove leaf oil, cocoa (Theobroma cacao) butter, coco-caprylate/caprate, coconut (Cocos nucifera) oil, collagen, collagen amino acids, corn (Zea mays) oil, free fatty acids, decyl oleate, dimethicone copolyol, dimethiconol, dioctyl adipate, dioctyl succinate, dipentaerythrityl hexacaprylate/hexacaprate, DNA, emu oil, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, Glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, Mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (Mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, pomegranate seed oil (Unica granatum), potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinol palmitate, rice (Oryza sativa) bran oil, RNA, rosemary (Rosmarinus officinalis) oil, rose oil, rosehip seed oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tea tree oil (Melaleuca alternifolia), tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

Antioxidants

In some embodiments, the topical composition comprises one or more antioxidant agents. Examples of antioxidants include acetyl cysteine, ascorbic acid polypeptide, ascorbyl dipalmitate, ascorbyl methylsilanol pectinate, ascorbyl palmitate, ascorbyl stearate, butylated hydroxyanisole (BHA), butylated hydroxytoluene (BHT), t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphit.

Nuclear Factor Erythroid-2-Related Factor 2 (Nrf2) Activators

In some embodiments, the topical composition comprises one or more Nrf2 activators. In some embodiments, the Nrf2 activators are isothiocyanate-containing components found in certain natural products. In some embodiments, the topical composition comprises Moringa oleifera leaf, seed, root, and/or stem extracts, powders, and/or oils. In some embodiments, the topical composition comprises Bixa orellana extracts, powders, and/or oils, including, for example, Annatto (bixin and norbixin). In some embodiments, the topical composition comprises Brassica oleracea extracts, powders, and/or oils from Brassica oleracea cultivars, including var. gemmifera, var. sebellica, var. italica, var. botrytis, var costata, var. acephala and var. capitata. In some embodiments, the topical composition comprises Eruca Vesicaria sativa powders, extracts, and/or oils. In some embodiments, the topical composition comprises Matricaria chamomilla extracts, powders, and/or oils. In some embodiments, the topical composition comprises Raphanus sativus extract, powders, and/or oil. In some embodiments, the topical composition comprises Vitis vinifera seed extract. In some embodiments, the topical composition comprises Curcumin longa extracts, powders, and/or oils. In some embodiments, for example, the topical composition comprises Camellia sinensis (green tea) extract.

Thickening Agents

In some embodiments, the topical composition comprises one or more thickening agents. As used herein, a thickening agent, or thickener, or gelling agent, includes one or more substances that can increase the viscosity of the composition. In some embodiments, thickeners increase the viscosity of the composition without substantially modifying the efficacy of the one or more active components within the composition. In some embodiments, thickeners increase the stability of the composition. In some embodiments, thickeners include hydrogenated polyisobutene or trihydroxystearin, or a mixture of both.

In various embodiments, thickening agents include carboxylic acid polymers, crosslinked polyacrylate polymers, polyacrylamide polymers, polysaccharides, and gums. Examples of crosslinked polyacrylate polymers include cationic and nonionic polymers.

Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Further examples of carboxylic acid polymers include carbomers, which are homopolymers of acrylic acid crosslinked with allyl ethers of sucrose or pentaerytritol.

Examples of polyacrylamide polymers (including nonionic polyacrylamide polymers having substituted, branched, or unbranched polymers) include polyacrylamide, isoparaffin and laureth-7, multi-block copolymers of acrylamides and substituted acrylamides with acrylic acids and substituted acrylic acids.

Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Further examples include an alkyl-substituted cellulose in which one or more of the hydroxy groups of the cellulose polymer is hydroxyalkylated (e.g., hydroxy ethylated or hydroxypropylated) to form a hydroxyalkylated cellulose that can then be modified with a C₁₀-C₃₀ straight chain or branched chain alkyl group through an ether linkage. In some embodiments, the polymers are ethers of C₁₀-C₃₀ straight or branched chain alcohols with hydroxyalkylcelluloses. Further examples of polysaccharides include scleroglucans comprising a linear chain of (1-3) linked glucose units with a (1-6) linked glucose every three unit.

Examples of gums include acacia, acacia Senegal, acacia Seyal, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, konjac gum, locust bean gum, natto gum, potassium alginate, potassium carrageenan, propylene glycol alginate, sclerotium gum, sodium carboxymethyl dextran, sodium carrageenan, solagum AX, taragum, tragacanth gum, xanthan gum, and mixtures thereof.

Emulsifying Agents

In some embodiments, the topical composition comprises one or more emulsifiers. As used herein, an emulsifier is an agent that can reduce the interfacial tension between phases and improve the formulation and stability of an emulsion. The emulsifiers can be nonionic, cationic, anionic, and zwitterionic emulsifiers. Examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, cetearyl alcohol, sorbitan olivate, cetearyl olivate, glyceryl caprylate, and mixtures thereof

Structuring Agents

In some embodiments, the topical composition comprises a structuring agent. As used herein, a structuring agent modifies the rheological characteristics of the composition to contribute to the composition's stability. In some embodiments, the structuring agent also functions as an emulsifier or surfactant. Examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof

Preservatives

In some embodiments, the topical composition comprises one or more preservatives. Examples of preservatives include quaternary ammonium preservatives, such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal, sodium levulinate, potassium sorbate, tetrasodium glutamate diacetate, or combinations thereof.

Silicone Compounds

In some embodiments, the topical composition comprises one or more silicone containing compounds. As used herein, a silicone containing compound includes any member of a family of polymeric products having a molecular backbone that is made up of alternating silicon and oxygen atoms with side groups attached to the silicon atoms. In such embodiments, the —Si—O— chain lengths, side groups, and crosslinking can be varied. As such, the silicone containing compounds can be synthesized into a wide variety of materials. They can vary in consistency from a liquid to gel form. Examples of silicone containing compounds include silicone oils (e.g., volatile and non-volatile oils), gels, and solids. In some embodiments, the silicon containing compounds includes a silicone oil such as a polyorganosiloxane. Examples of polyorganosiloxanes include dimethicone, cyclomethicone, polysilicone-11, phenyl trimethicone, trimethylsilylamodimethicone, stearoxytrimethylsilane, or mixtures of these and/or other organosiloxane materials in any given ratio to achieve the desired consistency and application characteristics depending upon the intended application (e.g., to a particular area such as the skin, hair, or eyes). As used herein, a “volatile silicone oil” includes a silicone oil having a low heat of vaporization, i.e., normally less than about 50 cal per gram of silicone oil. Examples of volatile silicone oils include cyclomethicones; low viscosity dimethicones, i.e., dimethicones having a viscosity of about 50 cst or less (e.g., dimethicones).

Essential Oils

In some embodiments, the topical composition comprises one or more essential oils. Essential oils include oils derived from herbs, flowers, trees, and other plants. Such oils can be present as tiny droplets between the plant's cells and can be extracted by methods known to those of skill in the art (e.g., steam distilled, enfleurage (i.e., extraction by using fat), maceration, solvent extraction, or mechanical pressing). When essential oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160 to 240° C. and densities ranging from about 0.759 to about 1.096.

Essential oils are generally named by the plant from which the oil is found. For example, rose oil or peppermint oil are derived from rose or peppermint plants, respectively. Examples of essential oils include sesame oil, Macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Botanical Extracts

In some embodiments, the topical composition comprises one or more botanical extracts. Botanical extracts are derived from various parts of herbs, trees, and other plants including, the flowers, stems, seeds, roots, and fruit thereof. In some embodiments, the extracts include one or more of the following: absinthium, aconite, agar, algin, allantoin, aloe vera, althea root, ambrette seed, angelica, anise, Arnica montana, asafetida, astragalus, avocado, lemon balm, barberry, basil sweet, bamboo (Bambusa vulgaris), bayberry bark, bee pollen, beeswax, benzoin, bilberry, bisabolol, blood root, Boswelia serrata, borage, buckthorn, buckwheat honey, burdock, cade oil, calamus, calendula, cananga oil, caraway, Cardamon, cascara sagrada, cascarilla bark, celery seed, chamomile, chaparral, chenpodium, chicory root, coatis (rhizoma coptidis), comfrey root, cortex phellodendri chinensis (amur cork tree bark) dandelion root, dictamnus root bark (cortex dictamni) echinacea, eleuthero, eucalyptus, evening primrose, fangfeng, fenugreek, feverfew, Fo-ti, ganoderma, genet, geranium (wild), ginko, ginseng, gotu kola, giant knotweed (rhizoma polygonie cuspidate) rhizome honey, hops, horse chestnut, horsetail, hypericum, Irish moss, job's tears, jojoba, jujube, licorice root, lycium fruit, magnolia flower, melatonin, neem, parsley, Pisum sativum (pea) seed extract, poria, quillaia, Radix sophorea flavescetis (light yellow spoor root), Rehmannia, rhoiola rosea root, rose hips, rosemary, rubarb root (rhizoma rhei), sage, sarsaparilla, sea kelp, tannic acid, thyme, willow bark, witch hazel, yarrow, and yucca.

Herbal Extracts

In some embodiments, the topical composition comprises one or more plant extracts. In some embodiments, the extracts are from plants used in traditional Chinese medicine for their healing, anti-swelling, wound healing, anti-inflammatory, or antihistaminic properties. In some embodiments, the herbs can include: atractylodes (baizhu and cangzhu), baizhu (atractylodes), bletilla tuber (baiji), cangzhu (atractylodes), dahurian angelica (baizhi), dittany bark (baixianpi), forsythia fruit (lianqiao), garden burnet (diyu), licorice (Glycyrrhiza glabra, including glycyrrhizin, glycyrrhizinic acid, isoliquiritin, glycyrrhizic acid), honeysuckle flower (jinyinhua), knotweed, giant (huzhang), ligusticum (gaoben), luffa (sigualuo), mume (smoked plum or wumei), pearl (zhenzhu or margarita), mother of pearl (zhenzhumu), peony bark and peony root (mudapi, shaoyao: chishaoyao and baishoyao), phellodendron bark (hungbai, purslane, common, machixian), red sage (danshen), safflower (false saffron: honghua), sichuan lovage (chuanxiog), skullcap, and baikal (huangqin).

Pharmaceutical Agents

In some embodiments, the topical composition comprises one or more pharmaceutical agents. Examples of pharmaceutical agents include existing agents used to treat dermatitis, analgesics, anesthetics, antihistamines, anti-inflammatory agents including non-steroidal anti-inflammatory drugs, antibiotics, antifungals, antivirals, antimicrobials, antipruritics, antipsoriatic agents, antiseborrheic agents, biologically active proteins and peptides, burn treatment agents, diaper rash treatment agents, skin protectant and/or barrier agents, corticosteroids (e.g., hydrocortisone), sunburn treatment agents, sunscreens, wound treatment agents, wound healing agents, and cannabinoids that mitigate itch sensation and inflammation.

In some embodiments, the topical composition comprises endocannabinoids (EC) or the bioactive lipids anandamide (AEA) or N-palmitoylethanolamine (PEA), which can decrease histamine-induced itch, decrease the release of the pro-inflammatory Th1 cytokine, and stimulate lipid production.

In some embodiments, the topical composition comprises one or more pharmaceutical agents that reduces skin inflammation through one or more biological mechanisms of action, including agents having activity for reducing inflammatory cytokine production in a cell, including IL-I, ILB, TNF-alpha, Il-8, Il-6, IL-17, Il-23, Il-22, Il-12, Il-4; inhibiting NF-KB pro-inflammatory signaling pathway; inhibiting mitogen-activated protein kinases; inhibiting JAK/STAT pro-inflammatory signaling; inhibiting histamine release from Mast cells; inhibiting COX2 and PGE2 production; suppressing transient receptor potential ankyrinin1 (TRPA1) signaling; and increasing NRF2 pathway activity.

Probiotics, Probiotic Derivatives, and Prebiotics

In some embodiments, the topical composition comprises one or more probiotics, probiotic derivatives, or prebiotics. In some embodiments, the probiotics, probiotic derivatives, or prebiotics are administered daily, including, for example, once per day, twice daily, three times per daily, etc. In some embodiments, the one or more probiotic strains can be selected from Lactobacillus brevis, Lactobacillus johnsonnii, Lactobacillus salibarius, Lactobacillus paracasei, Stretococcus thermophilus, Bifidobacterium longum, Roseomonas mucosa, Vitresoscilla filiformis, and kefir gel. In some embodiments, the prebiotic is Avena sativa (oat) kernel extract. In some embodiments, the one or more probiotics, probiotic derivatives, or prebiotics are formulated or administered with one or more compounds, including acetic acid, diacetyl (butane-2,3-dione), sphingomyelinase, hyaluronic acid, lipoteichoic acid and peptidoglycan, and lactic acid.

Cosmetic Components

In some embodiments, the topical composition comprises one or more cosmetic components. Examples include fragrances (artificial and natural), dyes and color components (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraminobenzoic acid (PABA) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), humectants (e.g., sorbitol, urea, and mannitol), magnesium and/or aluminum hydroxide stearate, and skin conditioning agents (e.g., aloe extracts, allantoin, bisabolol, dimethicone, hyaluronic acid, and dipotassium glycyrrhizate).

### Examples

The following examples are provided to aid in the understanding of the present disclosure, the true scope of which is set forth in the appended claims. One of skill in the art would appreciate that modifications can be made in the formulations and/or procedures set forth without departing from the spirit of the disclosure.

Topical Formulation Example 1.

| Component | Concentration |
|---|---|
| Aloe vera leaf juice | 56.845% |
| Jojoba oil | 12.0% |
| Sodium cocycl isethionate | 7.0% |
| Moringa oleifera leaf extract | 5.0% |
| Calendula flower extract (CO₂) | 0.3% |
| Xanthan gum | 1.0% |
| Nicotinamide | 2.0% |
| Glycerin | 3.0% |
| Green tea extract (ECGC) | 0.035% |
| triticum vulgare (wheat) seed extract (C + eramide) | 0.32% |
| Sunflower oil | 3.0% |
| Citric acid | 0.3% |
| Phytosterol | 0.2% |
| Benzyl alcohol, dehydroacetic acid (Geogard 221) | 1.0% |
| Squalane | 1.0% |
| Tocopheryl acetate | 1.0% |
| Cetearyl alcohol and sorbitan olivate | 3.0% |
| Moringa Oleifera, Eruca Sativa, Brassica Oleracea | 3.0% |
| var. Sabellica, and Brassica oleracea var. | |
| Italica extract | |

Topical Formulation Example 2.

| Component | Concentration |
|---|---|
| Water | 37.845% |
| Aloe vera | 15.0% |
| Jojoba oil | 12% |
| Sodium cocycl isethionate | 7.0% |
| Moringa oleifera Leaf extract | 5.0% |
| Licorice plant extract glycyrrhizinic acid | 1.0% |
| Avena Sativa (Oat) kernal extract | 1.0% |
| Glycerin | 5.0% |
| Green tea extract (ECGC) | 0.035% |
| Calendula flower extract (CO₂) | 0.3% |
| Xanthan gum | 1.0% |
| triticum vulgare (wheat) seed extract (C + eramide) | 0.32% |
| Sunflower oil | 2.0% |
| Citric acid | 0.3% |
| Coconut oil | 3.0% |
| Phytosterol | 0.2% |
| Benzyl alcohol, dehydroacetic acid (Geogard 221) | 1.0% |
| Squalane | 1.0% |
| Tocopheryl acetate | 1.0% |
| Cetearyl alcohol and sorbitan olivate | 3.0% |
| Moringa Oleifera, Eruca Sativa, Brassica Oleracea | 3.0% |
| var. Sabellica, and Brassica oleracea var. | |
| Italica extract | |

Topical Formulation Example 3.

| Component | Concentration |
|---|---|
| Water | 50.135% |
| Aloe vera leaf juice | 18.04% |
| Jojoba oil | 12% |
| Moringa oleifera Leaf extract | 1.0% |
| Glycerin | 5.0% |
| (Xanthan gum or solagum AX), (taragum or | 1.0% |
| solagum AX), Konjac gum | |
| triticum vulgare (wheat) seed extract (C + eramide) | 0.32% |
| Sunflower oil | 3.0% |
| Citric acid | 0.3% |
| Phytosterol | 0.2% |
| Benzyl alcohol, dehydroacetic acid (Geogard 221) | 1.0% |
| Squalane | 1.0% |
| Tocopheryl acetate | 1.0% |
| Cetearyl alcohol and sorbitan olivate | 3.0% |

Topical Formulation Example 4.

| Component | Concentration |
|---|---|
| Water | 37.845% |
| Aloe vera | 14.0% |
| Jojoba oil | 12% |
| Sodium cocycl isethionate | 7.0% |
| Moringa oleifera Leaf extract | 5.0% |
| Vegan silk polypeptide | 1.0% |
| Licorice plant extract glycyrrhizinic acid | 1.0% |
| Avena Sativa (Oat) kernal extract | 1.0% |
| Glycerin | 5.0% |
| Green tea extract (ECGC) | 0.035% |
| Calendula flower extract (CO₂) | 0.3% |
| Xanthan gum | 1.0% |
| triticum vulgare (wheat) seed extract (C + eramide) | 0.32% |
| Sunflower oil | 2.0% |
| Citric acid | 0.3% |
| Coconut oil | 3.0% |
| Phytosterol | 0.2% |
| Benzyl alcohol, dehydroacetic acid (Geogard 221) | 1.0% |
| Squalane | 1.0% |
| Tocopheryl acetate | 1.0% |
| Cetearyl alcohol and sorbitan olivate | 3.0% |
| Moringa Oleifera, Eruca Sativa, Brassica Oleracea | 3.0% |
| var. Sabellica, and Brassica oleracea var. | |
| Italica extract | |

Method of Administering Topical Composition to a Subject.

In various embodiments, a method of treating or preventing a skin condition comprising the topical application of a topical composition described herein is provided. In some embodiments, the topical composition comprises Moringa oleifera leaf extract, Moringa oleifera seed extract, Moringa oleifera root extract, Moringa oleifera stem extract, Moringa oleifera seed oil, Moringa oleifera leaf powder, or any combination thereof. In some embodiments, the topical application comprises the composition of Topical Formulation Example 1, or a derivative thereof, to treat the skin condition. In some embodiments, the topical application comprises the composition of Topical Formulation Example 2, or a derivative thereof, to treat the skin condition. In some embodiments, the topical application comprises the composition of Topical Formulation Example 3, or a derivative thereof, to treat the skin condition. In some embodiments, the topical application comprises the composition of Topical Formulation Example 4, or a derivative thereof, to treat the skin condition.

In this context, the term “derivative” refers to a modified version of Topical Formulation Example 1, Topical Formulation Example 2, Topical Formulation Example 3, or Topical Formulation Example 4, whereby the modification is directed to a change in concentration for one or components, the absence of one or more components, and/or the inclusion of one or more additional components.

In some embodiments, the method comprises applying a topical composition (e.g., Topical Formulation Example 1, Topical Formulation Example 2, Topical Formulation Example 3, or Topical Formulation Example 4, and derivatives thereof) to a portion of skin in need thereof (e.g., human skin having a skin condition), wherein the topical application reduces or prevents the skin condition when compared to another portion of skin in need thereof that has not been treated with the composition. Examples of skin conditions include dermatitis (including, but not limited to radiation dermatitis, atopic dermatitis, seborrheic dermatitis, nummular dermatitis, contact dermatitis, exfoliative dermatitis, perioral dermatitis, and stasis dermatitis), psoriasis, and other inflammatory skin conditions. In some embodiments, the skin condition can be caused by age, exposure to UV light, irradiation, chronic sun exposure, environmental pollutants, air pollution, wind, cold, heat, chemicals, disease pathologies, smoking, or lack of nutrition. The skin can be facial skin or non-facial skin (e.g., arms, legs, hands, chest, back, feet, etc.).

In some embodiments, the method further comprises identifying a subject in need of skin treatment. The subject can be a human male or female. The subject can be any age, including children aged 1-17, young adults aged 18-29, and adults aged between 30-110 years, including any specific age or subrange of ages therein. One of ordinary skill in the art would appreciate that the age and sex of the subject are factors that affect the pH of the subject's skin.

In some embodiments, the method comprises topically applying the topical composition in an amount effective to increase cellular anti-oxidant defense mechanisms (e.g., exogenous additions of anti-oxidants can bolster, replenish, or prevent the loss of cellular antioxidants such as catalase and glutathione in skin cells (e.g., keratinocytes, melanocytes, langerhans cells, etc.), which can reduce or prevent oxidative damage to the skin, cellular, proteins, and lipids). In some embodiments, the method comprises topically applying the topical composition in an amount effective to reduce or prevent oxidative damage to skin (including reducing the amount lipid peroxides and/or protein oxidation in the skin). In some embodiments, the topical composition can decrease the amount of internal oxidation and/or external oxidative damage in a cell by decreasing the pro-oxidant iNOS synthase enzyme.

In some embodiments, a method of treating erythema or reducing the appearance of symptoms associated with erythema, sensitive skin, or inflamed skin is provided. In some embodiments, the method comprises topically applying to erythemic, sensitive, or inflamed skin one or more of the compositions of disclosed throughout this specification (e.g., Topical Formulation Example 1, Topical Formulation Example 2, Topical Formulation Example 3, Topical Formulation Example 4, and derivatives thereof). In some embodiments, the composition comprises Moringa oleifera leaf extract, Moringa oleifera seed extract, Moringa oleifera root extract, Moringa oleifera stem extract, Moringa oleifera seed oil, Moringa oleifera leaf powder, or any combination thereof. The erythema can be caused by radiation, skin sunburn, electrical treatments of skin, skin burns, contact allergies, systemic allergies, skin toxicity, exercise, insect stings, bacterial infection, viral infection, fungal infection, protozoa infection, massage, or windburn.

Exemplary embodiments of the topical compositions and methods are described above in detail. The topical compositions and methods are not limited to the specific embodiments described herein, but rather, components of the topical compositions and/or steps of the method may be utilized independently and separately from other components and/or steps described herein. It is contemplated that any embodiment discussed in this specification can be implemented with respect to any method or composition of the disclosure, and vice versa. Furthermore, compositions of the disclosure can be used to achieve methods of the disclosure.

A recitation of ranges of values herein are merely intended to serve as a shorthand method of referring individually to each separate value falling within the range, unless otherwise indicated herein, and each separate value is incorporated into the specification as if it were individually recited herein. As will be understood by one skilled in the art, ranges disclosed herein encompass any and all possible subranges and combinations of subranges thereof. Any listed range can be easily recognized as sufficiently describing the same range being broken down into at least equal halves, thirds, quarters, fifths, tenths, etc. As a non-limiting example, each range discussed herein can be readily broken down into a lower third, middle third and upper third, etc. As will also be understood by one skilled in the art, language such as “up to,” “at least,” “greater than,” “less than,” and the like include the number recited and refer to ranges which can be subsequently broken down into subranges as discussed above. Finally, as will be understood by one skilled in the art, a range includes each endpoint and individual member. Thus, for example, a composition having 1-3 components refers to compositions having 1, 2, or 3 components. Similarly, a composition having 1-5 components refers to compositions having 1, 2, 3, 4, or 5 components, and so forth.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, the use of examples, or exemplary language (e.g., “such as”), is intended to illuminate the embodiments and does not pose a limitation on the scope of the claims unless otherwise stated. No language in the specification should be construed as indicating any non-claimed element as essential.

As used herein, the terms “about” and “substantially” will be understood by persons of ordinary skill in the art and will vary to some extent depending upon the context in which it is used. If there are uses of the term which are not clear to persons of ordinary skill in the art, given the context in which it is used, “about” and “substantially” will mean up to plus or minus 10% of the particular term.

As used herein, the terms “subject,” “individual,” or “patient” can be an individual organism, a vertebrate, a mammal, or a human. “Mammal” includes a human, non-human primate, murine (e.g., mouse, rat, guinea pig, hamster), ovine, bovine, ruminant, lagomorph, porcine, caprine, equine, canine, feline, avis, etc. In some embodiments herein, the mammal is human.

The term “administering” a composition to a subject means delivering the composition to the subject. “Administering” can include prophylactic administration of the composition (i.e., before one or more symptoms of dermatitis are detectable) and/or therapeutic administration of the composition (i.e., after the dermatitis or skin condition and/or one or more symptoms of dermatitis or skin condition are detectable). The methods of some embodiments may include administering one or more compounds, compositions, or agents. If more than one compound, composition, or agent is to be administered it may be administered together at substantially the same time, and/or be administered before, concomitantly with, and/or after administration of another composition or therapeutic procedure. All methods described herein can be performed in any suitable order unless otherwise indicated herein or otherwise clearly contradicted by context.

As used herein, the terms “effective amount” or “therapeutically effective amount,” refer to a quantity sufficient to achieve a desired therapeutic and/or prophylactic effect, e.g., an amount which results in the full or partial amelioration of disorders or symptoms, e.g., dermatitis, in a subject in need thereof. In the context of therapeutic or prophylactic applications, the amount of a composition administered to the subject will depend on the type and severity of the disorder or symptom and on the characteristics of the individual, such as general health, age, sex, body weight, and tolerance to drugs and nutraceuticals. It will also depend on the degree, severity, and type of disorder or symptom. The topical compositions can also be administered in combination with one or more additional compounds, compositions, drugs, supplements, or nutraceuticals. In some embodiments, multiple doses are administered. In some embodiments, multiple therapeutic compositions or compounds are administered. In the methods described herein, the topical compositions may be administered to a subject having one or more signs or symptoms of a disorder described herein.

This written description uses examples to disclose the present embodiments, including the best mode, and also to enable any person skilled in the art to practice the present embodiments, including making and using any topical compositions or performing any methods. The patentable scope of the present embodiments is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

I/We claim:

1. A composition formulated for topical application, the composition comprising:
a Moringa oleifera extract in an amount ranging from 0.1-6.0%;
a ceramide in an amount ranging from 0.1-1.0%;
a free fatty acid in an amount ranging from 0.05-8%;
a sterol in an amount ranging from 0.01-1.0%; and
a silk in an amount ranging from 0.05-5.0%;
wherein each respective amount is based on weight percent.

2. The composition of claim 1, further comprising a calendula flower extract in an amount ranging from 0.05-3.0%.

3. The composition of claim 1, wherein the ceramide is a Triticum vulgare (wheat) seed extract.

4. The composition of claim 1, wherein the sterol is cholesterol or phytosterol.

5. The composition of claim 1, wherein the silk is vegan silk polypeptide.

6. The composition of claim 1, wherein the free fatty acid is from an extract of sunflower oil or safflower oil.

7. The composition of claim 1, wherein the free fatty acid is linoleic acid, palmitic acid, stearic acid, or a combination thereof.

8. The composition of claim 1, further comprising a mixture of Moringa oleifera, Eruca sativa, Brassica oleracea var. Sabellica, and Brassica oleracea var. Italica extract in an amount ranging from 1.0-10.0%, wherein the mixture is a distinct component with respect to the Moringa oleifera extract.

9. The composition of claim 1, further comprising green tea extract in an amount ranging from 0.01-2.0%.

10. The composition of claim 1, further comprising glycyrrhizinic acid in an amount ranging from 0.2-3.0%.

11. The composition of claim 1, further comprising:
water in an amount ranging from 25-50%;
an aloe vera in an amount ranging from 5-20%;
a jojoba oil in an amount ranging from 5-18%;
a sodium cocycl isethionate in an amount ranging from 2-11%;
a glycerin in an amount ranging from 1-10%;
a gum in an amount ranging from 0.2-3%;
Avena sativa (oat) kernel extract in an amount ranging from 0.2-3.0%
a citric acid in an amount ranging from 0.09-2%;
a coconut oil in an amount ranging from 0.9-6%;
a mixture of benzyl alcohol and dehydroacetic acid in a combined amount ranging from 0.5-3.0%;
a squalane in an amount ranging from 0.2-3%;
tocopheryl acetate in an amount ranging from 0.2-3.0%; and
a mixture of cetearyl alcohol and sorbitan olivate in a combined amount ranging from 0.2-6.0%.

12. A topical composition formulated for application to human skin, the topical composition comprising:
a Moringa oleifera extract in an amount ranging from 1.0-10.0%;
a silk in an amount ranging from 0.09-3.0%;
a sterol selected from the group of cholesterol and phytosterol in an amount ranging from 0.05-1.0%;
a mixture of Moringa oleifera, Eruca sativa, Brassica oleracea var. Sabellica, and Brassica oleracea var. Italica extract in an amount ranging from 1.0-10.0%, wherein the mixture is a distinct component with respect to the Moringa oleifera extract;
a Triticum vulgare (wheat) seed extract in an amount ranging from 0.1-0.7%;
a sunflower oil in an amount ranging from 0.5-5%;
a green tea extract in an amount ranging from 0.01-2%;
a glycyrrhizinic acid in an amount ranging from 0.2-3%;
an Avena sativa (oat) kernel extract in an amount ranging from 0.2-3%;
water in an amount ranging from 25-50%;
an aloe vera in an amount ranging from 5-20%;
a jojoba oil in an amount ranging from 5-18%;
a sodium cocycl isethionate in an amount ranging from 2-11%;
a glycerin in an amount ranging from 1-10%;
a xanthan gum in an amount ranging from 0.2-3%;
a citric acid in an amount ranging from 0.09-2%;
a coconut oil in an amount ranging from 0.9-6%;
a benzyl alcohol and dehydroacetic acid in a combined amount ranging from 0.5-3%;
a squalane in an amount ranging from 0.2-3%;
a tocopheryl acetate in an amount ranging from 0.2-3%; and
a mixture of cetearyl alcohol and sorbitan olivate in a combined amount ranging from 0.2-6%;
wherein each respective amount is based on weight percent.

13. The composition of claim 12, wherein
the Moringa oleifera extract is in an amount ranging from 2.0-7.0%;
the silk is in an amount ranging from 0.7-1.0%;
the sterol is in an amount ranging from 0.09-1.0%;
the mixture of Moringa oleifera, Eruca sativa, Brassica oleracea var. Sabellica, and Brassica oleracea var. Italica extract is in an amount ranging from 1.5-6.0%;
the green tea extract is in an amount ranging from 0.01-1.0%;
the Triticum vulgare (wheat) seed extract is in an amount ranging from 0.1-0.7%;
the glycyrrhizinic acid is in an amount ranging from 0.5-2.5%;
the Avena sativa (oat) kernel extract is in an amount ranging from 0.5-2.5%;
the water is in an amount ranging from 30-45%;
the aloe vera is in an amount ranging from 8-18%;
the jojoba oil is in an amount ranging from 8-15%;
the sodium cocycl isethionate is in an amount ranging from 4-10%;
the glycerin is in an amount ranging from 3-8%;
the xanthan gum is in an amount ranging from 0.5-2.5%;
the sunflower oil is in an amount ranging from 0.5-4%;
the citric acid is in an amount ranging from 0.09-2%;
the coconut oil is in an amount ranging from 1-7%;
the mixture of benzyl alcohol and dehydroacetic acid is in a combined amount ranging from 0.5-2.5%;
the squalane in an amount ranging from 0.5-2.5%;
the tocopheryl acetate is in an amount ranging from 0.5-2.5%; and
the mixture of cetearyl alcohol and sorbitan olivate is in a combined amount ranging from 1-5.5%;
wherein each respective amount is based on weight percent.

14. The composition of claim 12, wherein
the Moringa oleifera extract is in an amount ranging from 3.0-6.0%;
the silk is in an amount ranging from 0.8-1.5%;
the sterol is in an amount ranging from 0.15-0.7%;
the mixture of Moringa oleifera, Eruca sativa, Brassica oleracea var. Sabellica, and Brassica oleracea var. Italica extract is in an amount ranging from 2.0-5.0%;
the green tea extract is in an amount ranging from 0.02-0.2%;
the Triticum vulgare (wheat) seed extract is in an amount ranging from 0.2-0.5%;
the glycyrrhizinic acid is in an amount ranging from 0.5-1.5%;
the Avena sativa (oat) kernel extract is in an amount ranging from 0.5-1.5%;
the water is in an amount ranging from 35-40%;
the aloe vera is in an amount ranging from 11-17%;
the jojoba oil is in an amount ranging from 10-14%;
the sodium cocycl isethionate is in an amount ranging from 5-9%;
the glycerin is in an amount ranging from 3-7%;
the xanthan gum is in an amount ranging from 0.5-1.5%;
the sunflower oil is in an amount ranging from 1-3%;
the citric acid is in an amount ranging from 0.1-0.8%;
the coconut oil is in an amount ranging from 2-5%;
the mixture of benzyl alcohol and dehydroacetic acid is in a combined amount ranging from 0.5-1.5%;
the squalane is in an amount ranging from 0.5-1.5%;
the tocopheryl acetate is in an amount ranging from 0.5-1.5%; and
the mixture of cetearyl alcohol and sorbitan olivate is in a combined amount ranging from 2-5%.

15. The composition of claim 12, further comprising a calendula flower extract in an amount ranging from 0.05-1.0%.

16. A method of treating a skin disorder in a subject in need thereof, the method comprising:
topically administering to the subject a composition comprising:
a Moringa oleifera extract in an amount ranging from 0.1-6.0%;
a ceramide in an amount ranging from 0.1-1.0%;
a free fatty acid in an amount ranging from 0.05-8%;
a sterol in an amount ranging from 0.01-1.0%; and
a silk in an amount ranging from 0.05-5.0%;
wherein each respective amount is based on weight percent.

17. The method of claim 16, wherein the composition further comprises:
a mixture of Moringa oleifera, Eruca sativa, Brassica oleracea var. Sabellica, and Brassica oleracea var. Italica extract in an amount ranging from 1.0-10.0%, wherein the mixture is a distinct component with respect to the Moringa oleifera extract;
a green tea extract in an amount ranging from 0.01-2.0%;
a glycyrrhizinic acid in an amount ranging from 0.2-3%;
an Avena sativa (oat) kernel extract in an amount ranging from 0.2-3%;
a water in an amount ranging from 25-50%;
an aloe vera in an amount ranging from 5-20%;
a jojoba oil in an amount ranging from 5-18%;
a sodium cocycl isethionate in an amount ranging from 2-11%;
a glycerin in an amount ranging from 1-10%;
a xanthan gum in an amount ranging from 0.2-3%;
a citric acid in an amount ranging from 0.09-2%;
a coconut oil in an amount ranging from 0.9-6%;
a mixture of benzyl alcohol and dehydroacetic acid in a combined amount ranging from 0.5-3%;
a squalane in an amount ranging from 0.2-3%;
an atocopheryl acetate in an amount ranging from 0.2-3%; and
a mixture of cetearyl alcohol and sorbitan olivate in a combined amount ranging from 0.2-4.5%.

18. The method of claim 16, wherein the skin disorder is radiation dermatitis or erythema.

19. The method of claim 16, wherein the administering is completed once per day, twice per day, or three times per day.

20. The method of claim 16, wherein the composition is in the form of a cream or lotion.
